SEQUENCE LISTING (4 sequences)

SEQ ID NO: 1 - DNA, 2373 bp, synthetic construct
  source 1..2373: mol_type = other DNA
atggctcccaacctcagcaagaagacgcctgctggcctcctcggcgacgaggtggccccggtggagggactcaagccgtcgcggttcacccttaagggcaaggacctggccgtggacgggcacccggtcctgctggacgtgccggccaacatccgtctcaccccggcgtcgacgctcgtgcccgccgcggacgtccccgcagcgggcggcggcagcttcctcggcttcgacgcggcggcggccgagagccggcacgtggtgcccgtcggaaagctccgtgacattcggttcatgagcatcttccgtttcaaggtgtggtggacgacgcactgggtgggggacagcggcagggacgtggagaacgagacgcagatgatggtgctcgaccgctccgccggcgagcccggcggcggcggccgaccctacgtgctgctgctccccatcatcgagggctcgttccgggcctgcctcgaggccgggaaggtggaagactacgtggacctgtgcgtggagagcgggtcgtcggcggtgcgcggcgccgcgttccggagctcgctgtacctgcacgcgggcgacgacccgttcgagctcgtcgcggacgccgtcagggtggtccgtgcgcacctgggcacgttccggaccatggaggagaagacgccgccgccgatcgtggacaagttcgggtggtgcacgtgggacgccttctacctcaaggtgcacccggagggcgtgtgggagggcgtgcgccgcctggcggagggcggctgcccgccggggctggtgctcatcgacgacggctggcagtccatctgccacgacgaggacgacccgaacagcggcgaggagggcatgaaccgcacctccgccggcgagcagatgccctgccgcctcatcaagttccaggagaaccacaagttcagggagtacaagcagggcgggatgggcgcgttcgtgcgggagatgaaggcggcgttccccaccgtggagcaggtgtacgtgtggcacgcgctgtgcgggtactggggcggcctccgccccggcgcgcccggcctgccgcccgccaaggtggtggcgcccaagctctcccccggcctgcagcgcaccatggaggacctcgccgtcgacaagatcgtcaacaacggtgtcggcctcgtcgaccccaagcgcgcgcacgagctctacgatggtttgcactcccacctccaggcctccggcatcgacggcgtcaaggtcgacgtcattcacttgctggagatgctgtgcgaggagtacggcggccgtgtcgagctggccaaggcctacttcgccgggctgacggcgtcggtgcggcggcacttcggcggcaacggcgtgatcgcgagcatggagcactgcaacgacttcatgctgctgggcacggaggcggtggcgctgggccgcgtgggcgacgacttctggtgcacggacccctccggcgaccccaacggcaccttctggctgcaggggtgccacatggtgcactgcgcctacaactcgctgtggatgggcaacttcatccacccggactgggacatgttccagtccacgcacccctgcgccgccttccacgccgcgtcccgcgccatctccggcgggcccatctacgtcagcgactcggtggggcagcacgacttcgcgctgctccgccgcctggcgctccccgacggcaccgtcctccggtgcgagggccacgcgctgcccacgcgcgactgcctcttcgccgacccgctccacgacggccggaccgtgctcaagatctggaacgtgaaccgcttcgccggcgtcgtcggcgccttcaactgccagggcggcgggtggagccccgaggcgcggcggaacaagtgcttctcggagttctccgtgcccctggccgcgcgcgcctcgccgtccgacgtcgaatggaagagcggcaaagcgggaccaggcgtcagcgtcaagggcgtctcccagttcgccgtgtacgcggtcgaggccaggacgctgcagctgctgcgccccgacgagggcgtcgacctcacgctgcagcccttcacctacgagctcttcgtcgtcgcccccgtgcgcgtcatctcgcacgagcgggccatcaagttcgcgcccatcggactcgccaacatgctcaacaccgccggcgccgtgcaggcgttcgaggccaagaaagatgctagcggcgtcacggcagaggtgttcgtgaagggcgcaggggagctggtggcgtactcgtcggcgacgcccaggctctgcaaggtgaacggcgacgaggccgagttcacgtacaaggacggcgtggtcaccgtcgacgtgccgtggtcggggtcgtcgtcgaagctgtgtcgcgtccagtacgtctactga

SEQ ID NO: 2 - protein, 790 aa, synthetic construct
MAPNLSKKTPAGLLGDEVAPVEGLKPSRFTLKGKDLAVDGHPVLLDVPANIRLTPASTLVPAADVPAAGGGSFLGFDAAAAESRHVVPVGKLRDIRFMSIFRFKVWWTTHWVGDSGRDVENETQMMVLDRSAGEPGGGGRPYVLLLPIIEGSFRACLEAGKVEDYVDLCVESGSSAVRGAAFRSSLYLHAGDDPFELVADAVRVVRAHLGTFRTMEEKTPPPIVDKFGWCTWDAFYLKVHPEGVWEGVRRLAEGGCPPGLVLIDDGWQSICHDEDDPNSGEEGMNRTSAGEQMPCRLIKFQENHKFREYKQGGMGAFVREMKAAFPTVEQVYVWHALCGYWGGLRPGAPGLPPAKVVAPKLSPGLQRTMEDLAVDKIVNNGVGLVDPKRAHELYDGLHSHLQASGIDGVKVDVIHLLEMLCEEYGGRVELAKAYFAGLTASVRRHFGGNGVIASMEHCNDFMLLGTEAVALGRVGDDFWCTDPSGDPNGTFWLQGCHMVHCAYNSLWMGNFIHPDWDMFQSTHPCAAFHAASRAISGGPIYVSDSVGQHDFALLRRLALPDGTVLRCEGHALPTRDCLFADPLHDGRTVLKIWNVNRFAGVVGAFNCQGGGWSPEARRNKCFSEFSVPLAARASPSDVEWKSGKAGPGVSVKGVSQFAVYAVEARTLQLLRPDEGVDLTLQPFTYELFVVAPVRVISHERAIKFAPIGLANMLNTAGAVQAFEAKKDASGVTAEVFVKGAGELVAYSSATPRLCKVNGDEAEFTYKDGVVTVDVPWSGSSSKLCRVQYVY

SEQ ID NO: 3 - DNA, 33 bp, synthetic construct
  source 1..33: mol_type = other DNA
cgcggatccatggctcccaccaccagccaagag

SEQ ID NO: 4 - DNA, 47 bp, synthetic construct
  source 1..47: mol_type = other DNA
tgcttagaggtagagagagagagagagacgactgagggacgacagag

(19) United States
(12) Patent Application Publication
(10) Pub. No.: US 2024/0035043 A1
(43) Pub. Date: Feb. 1, 2024

(54) Application of maize ZmRAFS gene to improve crop heat stress tolerance

(71) Applicant: Northwest Agriculture and Forestry University, Xianyang city (CN)

(72) Inventors: TianYong Zhao, Xianyang city (CN); Ying Liu, Xianyang city (CN); Dong Yan, Xianyang city (CN); Yumin Zhang, Xianyang city (CN)

(21) Appl. No.: 18/225,899

(22) Filed: Jul. 25, 2023

Related U.S. Application Data
(63) Continuation of application No. PCT/CN2023/093524, filed on May 11, 2023.

(30) Foreign Application Priority Data
Jul. 27, 2022 (CN) 202210891677.3

(51) Int. Cl. C12N 15/82; C12N 9/10

(52) U.S. Cl. CPC C12N 15/8273; C12N 9/1051; C12Y 204/01082

(57) ABSTRACT

An application of maize raffinose synthase (ZrnRAFS) gene in maize to improve heat stress tolerance of crops is provided. The nucleotide sequence of the ZrnRAFS gene is shown in SEQ ID NO: 1, and the protein sequence encoded by the ZrnRAFS gene is shown in SEQ ID NO: 2. The ZrnRAFS gene is overexpressed in plants, raffinose is synthesized by using sucrose and galactinol, which increases the content of raffinose in maize leaves and improves the heat stress tolerance of maize.

### TECHNICAL FIELD

The disclosure relates to the field of plant biotechnology, and more particularly to a key enzyme gene ZmRAFS (GRMZM2G150906) encoding raffinose synthase that are responsible for synthesis of raffinose and an application/use thereof.

### STATEMENT REGARDING SEQUENCE LISTING

The sequence listing associated with this application is provided in text format in lieu of a paper copy and is hereby incorporated by reference into the specification. The name of the XML file containing the sequence listing is 23060TBYX-USP1-SL.xml. The XML file is 7,577 bytes; is created on Jul. 24, 2023; and is being submitted electronically via EFS-Web.

### BACKGROUND

A previous research achievement of the inventor discloses that overexpression of maize raffinose synthase (ZmRAFS) gene from Maize (also referred to as Zea mays) in Arabidopsis thaliana improves plant drought tolerance by hydrolyzing galactinol to produce inositol (Li, T., et al., Raffinose synthase enhances drought tolerance through raffinose synthesis or galactinol hydrolysis in maize and Arabidopsis plants. Journal of Biological Chemistry. 2020. 295:8064-8077.). Drought tolerance is the ability of a plant to survive or grow in an environment adapted to growth temperature (e.g., around 28° C.), under short-term or long-term water shortage.

### SUMMARY

The inventor finds that overexpression of maize raffinose synthase (ZmRAFS) gene (GRMZM2G150906) in maize plants increases the content of raffinose in leaves and enhances the heat stress tolerance of the plants.

Based on this, the disclosure provides an application of ZmRAFS gene in maize to improve heat stress tolerance of crops, and the sequence of the ZmRAFS gene is shown in SEQ ID NO: 1. The protein sequence encoded by the ZmRAFS gene is shown in SEQ ID NO: 2.

In some embodiments, the enzyme encoded by the ZmRAFS gene uses galactinol and sucrose as substrates to synthesize raffinose in the leaves, and the heat stress tolerance of the plants is improved by regulating the content of raffinose in the leaves. In an embodiment, the crop is maize.

### BRIEF DESCRIPTION OF DRAWINGS

FIG. 1 illustrates a schematic diagram of a maize overexpression vector; where pUbi represents a Maize Ubiquitin promoter, ZmRAFS represents a maize raffinose synthase gene, NOS represents a terminator, p35S represents a Cauliflower mosaic virus (CaMV) 35S promoter, Bar represents an herbicide-resistance gene, Tvsp represents another terminator, and HindIII and EcoRV represent restrictive enzyme sites.

FIGS. 2A-2D illustrate that the content of raffinose in leaves of maize plants overexpressing ZmRAFS is significantly increased. Specifically, FIG. 2A illustrates identification of overexpressed maize plants at genomic level; where Marker represents DNA molecular weight, Control represents polymerase chain reaction (PCR) water control (also referred to as blank control), Zong31 represents maize Zong31 inbred line genome, Plasmid represents Plasmid DNA, used as a positive control, PTF101.1 ZmRAFS (1 . . . 9) represent 9 independent transformation events overexpressing ZmRAFS. FIG. 2B illustrates identification of ZmRAFS mRNA level in leaves of overexpressed maize plants. FIG. 2C illustrates identification of ZmRAFS protein level in the leaves of the overexpressed maize plants; where Zong31 represents maize Zong31 inbred line, ZmRAFS #1, #2, #3 represent three plants overexpressing the ZmRAFS gene (background of Zong31), ZmGAPDH represents glyceraldehyde-3-phosphate dehydrogenase gene, as an internal reference. FIG. 2D illustrates determination of the content of raffinose in the leaves of the overexpressed maize plants; where Zong31 represents maize Zong31 inbred line, ZmRAFS #1, #2, #3 represent three plants overexpres sing the ZmRAFS gene (background of Zong31), data represents mean±standard error, n=3, and * represents significant difference (Student's t-test, * P<0.05, ** P<0.01).

FIGS. 3A-3C illustrate that overexpression of ZmRAFS gene significantly improves heat stress tolerance of the maize plants. Specifically, FIG. 3A illustrates growth phenotypes of maize plants overexpres sing the ZmRAFS gene and control plants under normal, heat shock treatment, and recovery, with a scale of 5 centimeters (cm). FIG. 3B illustrates that the survival rate of the maize plants overexpres sing the ZmRAFS gene after heat stress treatment is significantly higher than that of the control plants. FIG. 3C illustrates that the conductivity of the maize plants overexpres sing the ZmRAFS gene is significantly lower than that of the control plants after heat stress treatment; where Zong31 represents maize Zong31 inbred line; ZmRAFS (#1, #2, #3) represent ZmRAFS transgenic plants, data represents mean±standard error, each black dot represents one biological repeat (8 seedlings), and each plant has eight biological repeats, and different small letters represent significant differences (Duncan test, P<0.05).

### DETAILED DESCRIPTION OF EMBODIMENTS

Unless otherwise specified, scientific and technological terms and methods herein are understood by those skilled in the related art or are implemented by methods well-known to those skilled in the related art. The heat stress tolerance is the ability of plants to survive or grow at a high temperature (above 30° C.). The technical solution of the disclosure will be further described below with reference to the accompanying drawings and specific embodiments. Unless otherwise specified, the materials or reagents used in the following embodiments are all commercially available products.

### Embodiment

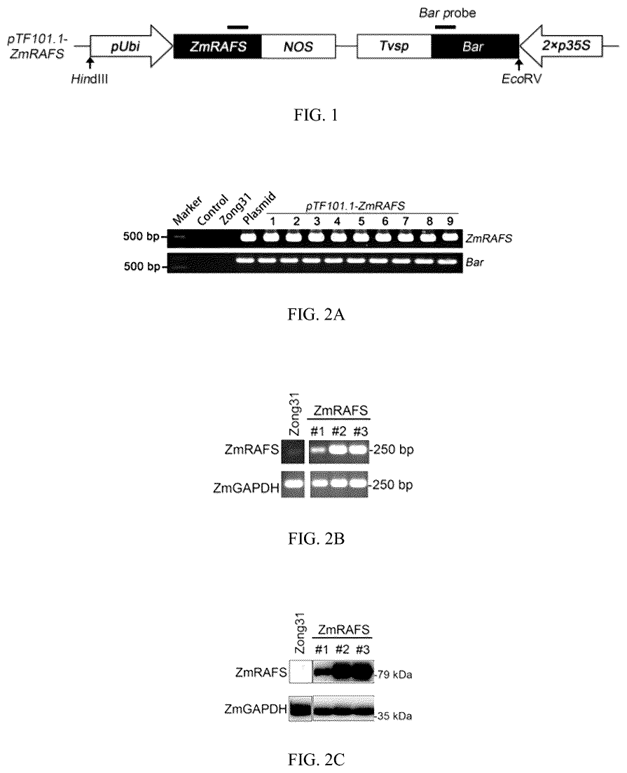

In this embodiment, the coding region of maize ZrnRAFS gene is cloned by polymerase chain reaction (PCR), a plant expression vector of the ZmRAFS gene is constructed, maize is transformed, and a maize transgenic plant is obtained. The method is specifically described as follows.
(1) RNA is extracted from leaves of B73 maize seedlings at the three-leaf stage and reverse transcribed into complementary DNA (cDNA).
(2) Using cDNA as a template, the coding region of ZmRAFS shown in SEQ ID NO: 1 is amplified with an upstream primer 5′ -CGCGGATCCATGGCTCCCACCACCAGCCAAGAG-3′ as shown in SEQ ID NO: 3 and a downstream primer 5′-TGCTTAGAGGTAGAGAGAGAGAGAGAGACGACTGAGGGACGACAGAG-3′ as shown in SEQ ID NO: 4. The amplification procedures are: pre-denaturation at 95° C. for 5 minutes; 95° C. denaturation for 30 seconds, annealing at 60° C. for 30 seconds, extension at 72° C. for 1 minute and 20 seconds, 35 cycles; and final extension at 72° C. for 10 minutes.
(3) The amplification product is purified, digested and recovered, and then ligated to the maize expression vector pTF101. After that, the vector is transformed into an Agrobacterium AGL1 strain, as shown in FIG. 1.
(4) The obtained Agrobacterium AGL1 strain is used for genetic transformation of maize, and the maize transformation receptor is the Zong31 inbred line (Hui Yang, Guoying Wang and Jingrui Dai, 2001, Transformation of maize inbred line Zong3 and Zong31, Journal of Agricultural Biotechnology, 9(04):334-337.doi:10.3969/j.issn.1674-7968.2001.04.008). Nine transgenic positive plants are obtained by infecting immature embryos of Zong31 at 10 days after pollination.

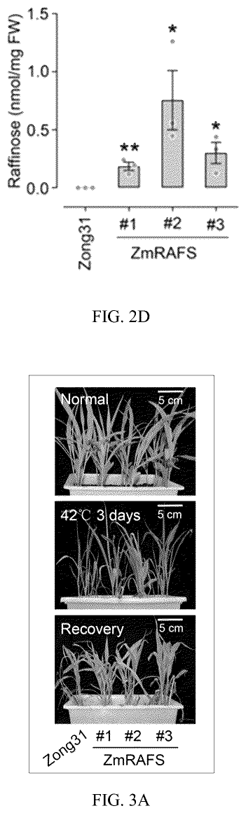

In addition, molecular characterization of three of the above nine transgenic positive plants (#1, #2 and #3) is performed. It is found that the expression of mRNA and protein of the ZmRAFS gene in the leaves of the transgenic plants is increased compared with that of the control, and the content of raffinose is significantly higher than that of the control. The method is specifically described as follows.
(a) The insertion and the mRNA expression of the transgene of the transgenic positive plants are identified by PCR and RT-PCR. The PCR amplification procedures are: pre-denaturation at 95° C. for 5 minutes; denaturation at 95° C. for 30 seconds, annealing at 60° C. for 30 seconds, extension at 72° C. for 30 seconds, 28 cycles; and final extension at 72° C. for 8 minutes; and the results are shown in FIGS. 2A and 2B.
(b) The protein accumulation of the ZmRAFS of the transgenic positive plants is identified by Western blotting, the procedures of Western blotting refer to the method published in the literature: Gu, L., et al., ZmGOLS2, a target of transcription factor ZmDREB2A, offers similar protection against abiotic stress as ZmDREB2A. Plant Mol Biol, 2016.90(1-2):p. 157-70, and the ZmRAFS primary antibody used in the method is prepared according to the method published in the literature: Li, T., et al., Regulation of seed vigor by manipulation of raffinose family oligosaccharides (RFOs) in maize and Arabidopsis. Molecular Plant. 2017. 10(12): 1540-1555, and the dilution ratio is 1: 5000. The secondary antibody (goat anti-rabbit IgG) is purchased from Jiangsu Cowin Biotech Co., Ltd, and the dilution ratio is 1: 10000. The results are shown in FIG. 2C.
(c) The content of raffinose in the leaves of the above plants #1, #2, #3 and a wild-type control plant is identified by high-performance liquid chromatography (HPLC), the method refers to the literature (Li, T., et al., Raffinose synthase enhances drought tolerance through raffinose synthesis or galactinol hydrolysis in maize and Arabidopsis plants. Journal of Biological Chemistry. 020. 295:8064-8077); and the results are shown in FIG. 2D.

SEQ ID NO: 1 is as follows:
atggctccca acctcagcaa gaagacgcct gctggcctcc tcggcgacga ggtggccccg 60
gtggagggac tcaagccgtc gcggttcacc cttaagggca aggacctggc cgtggacggg 120
cacccggtcc tgctggacgt gccggccaac atccgtctca ccccggcgtc gacgctcgtg 180
cccgccgcgg acgtccccgc agcgggcggc ggcagcttcc tcggcttcga cgcggcggcg 240
gccgagagcc ggcacgtggt gcccgtcgga aagctccgtg acattcggtt catgagcatc 300
ttccgtttca aggtgtggtg gacgacgcac tgggtggggg acagcggcag ggacgtggag 360
aacgagacgc agatgatggt gctcgaccgc tccgccggcg agcccggcgg cggcggccga 420
ccctacgtgc tgctgctccc catcatcgag ggctcgttcc gggcctgcct cgaggccggg 480
aaggtggaag actacgtgga cctgtgcgtg gagagcgggt cgtcggcggt gcgcggcgcc 540
gcgttccgga gctcgctgta cctgcacgcg ggcgacgacc cgttcgagct cgtcgcggac 600
gccgtcaggg tggtccgtgc gcacctgggc acgttccgga ccatggagga gaagacgccg 660
ccgccgatcg tggacaagtt cgggtggtgc acgtgggacg ccttctacct caaggtgcac 720
ccggagggcg tgtgggaggg cgtgcgccgc ctggcggagg gcggctgccc gccggggctg 780
gtgctcatcg acgacggctg gcagtccatc tgccacgacg aggacgaccc gaacagcggc 840
gaggagggca tgaaccgcac ctccgccggc gagcagatgc cctgccgcct catcaagttc 900
caggagaacc acaagttcag ggagtacaag cagggcggga tgggcgcgtt cgtgcgggag 960
atgaaggcgg cgttccccac cgtggagcag gtgtacgtgt ggcacgcgct gtgcgggtac 1020
tggggcggcc tccgccccgg cgcgcccggc ctgccgcccg ccaaggtggt ggcgcccaag 1080
ctctcccccg gcctgcagcg caccatggag gacctcgccg tcgacaagat cgtcaacaac 1140
ggtgtcggcc tcgtcgaccc caagcgcgcg cacgagctct acgatggttt gcactcccac 1200
ctccaggcct ccggcatcga cggcgtcaag gtcgacgtca ttcacttgct ggagatgctg 1260
tgcgaggagt acggcggccg tgtcgagctg gccaaggcct acttcgccgg gctgacggcg 1320
tcggtgcggc ggcacttcgg cggcaacggc gtgatcgcga gcatggagca ctgcaacgac 1380
ttcatgctgc tgggcacgga ggcggtggcg ctgggccgcg tgggcgacga cttctggtgc 1440
acggacccct ccggcgaccc caacggcacc ttctggctgc aggggtgcca catggtgcac 1500
tgcgcctaca actcgctgtg gatgggcaac ttcatccacc cggactggga catgttccag 1560
tccacgcacc cctgcgccgc cttccacgcc gcgtcccgcg ccatctccgg cgggcccatc 1620
tacgtcagcg actcggtggg gcagcacgac ttcgcgctgc tccgccgcct ggcgctcccc 1680
gacggcaccg tcctccggtg cgagggccac gcgctgccca cgcgcgactg cctcttcgcc 1740
gacccgctcc acgacggccg gaccgtgctc aagatctgga acgtgaaccg cttcgccggc 1800
gtcgtcggcg ccttcaactg ccagggcggc gggtggagcc ccgaggcgcg gcggaacaag 1860
tgcttctcgg agttctccgt gcccctggcc gcgcgcgcct cgccgtccga cgtcgaatgg 1920
aagagcggca aagcgggacc aggcgtcagc gtcaagggcg tctcccagtt cgccgtgtac 1980
gcggtcgagg ccaggacgct gcagctgctg cgccccgacg agggcgtcga cctcacgctg 2040
cagcccttca cctacgagct cttcgtcgtc gcccccgtgc gcgtcatctc gcacgagcgg 2100
gccatcaagt tcgcgcccat cggactcgcc aacatgctca acaccgccgg cgccgtgcag 2160
gcgttcgagg ccaagaaaga tgctagcggc gtcacggcag aggtgttcgt gaagggcgca 2220
ggggagctgg tggcgtactc gtcggcgacg cccaggctct gcaaggtgaa cggcgacgag 2280
gccgagttca cgtacaagga cggcgtggtc accgtcgacg tgccgtggtc ggggtcgtcg 2340
tcgaagctgt gtcgcgtcca gtacgtctac tga. 2373
SEQ ID NO: 2 is as follows:
MAPNLSKKTPAGLLGDEVAPVEGLKPSRFTLKGKDLAVDGH
PVLLDVPANIRLTPASTLVPAADVPAAGGGSFLGFDAAAA
ESRHVVPVGKLRDIRFMSIFRFKVWWTTHWVGDSGRDVEN
ETQMMVLDRSAGEPGGGGRPYVLLLPIIEGSFRACLEAGK
VEDYVDLCVESGSSAVRGAAFRSSLYLHAGDDPFELVADA
VRVVRAHLGTFRTMEEKTPPPIVDKFGWCTWDAFYLKVHP
EGVWEGVRRLAEGGCPPGLVLIDDGWQSICHDEDDPNSGE
EGMNRTSAGEQMPCRLIKFQENHKFREYKQGGMGAFVREM
KAAFPTVEQVYVWHALCGYWGGLRPGAPGLPPAKVVAPKL
SPGLQRTMEDLAVDKIVNNGVGLVDPKRAHELYDGLHSHL
QASGIDGVKVDVIHLLEMLCEEYGGRVELAKAYFAGLTAS
VRRHFGGNGVIASMEHCNDFMLLGTEAVALGRVGDDFWCT
DPSGDPNGTFWLQGCHMVHCAYNSLWMGNFIHPDWDMFQS
THPCAAFHAASRAISGGPIYVSDSVGQHDFALLRRLALPD
GTVLRCEGHALPTRDCLFADPLHDGRTVLKIWNVNRFAGV
VGAFNCQGGGWSPEARRNKCFSEFSVPLAARASPSDVEWK
SGKAGPGVSVKGVSQFAVYAVEARTLQLLRPDEGVDLTLQ
PFTYELFVVAPVRVISHERAIKFAPIGLANMLNTAGAVQA
FEAKKDASGVTAEVFVKGAGELVAYSSATPRLCKVNGDEA
EFTYKDGVVTVDVPWSGSSSKLCRVQYVY.

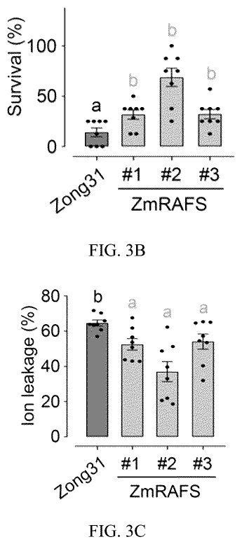

The specific method for identifying heat stress tolerance of the transgenic positive plants obtained from the embodiment is as follows.
(i) Maize seedling cultivation method is as follows. The ZmRAFS transgenic plants (#1, #2, #3) and the wild-type control plant (Zong31) are planted in an incubator. Seeds of each experimental group are soaked and germinated in darkness at 28° C. for 72 hours, the maize with the same growth vigor (root length of about 2-3 cm) is selected and transplanted into the pot, with 8 plants in each pot. When transplanting, the nutrient soil and water are mixed in a weight ratio of 1:1, the weight of each pot is controlled to 170 grams (g). After marking, the maize is placed in the incubator and grown in darkness at 28° C. for 16 hours under light (10,000 LUX)/8 hours.
(ii) Seedling heat shock treatment and survival rate statistics are as follows. When the seedlings reach the three-leaf stage, they are subjected to heat shock treatment at 42° C. When the leaves of the control group are irreversibly rolled (the rolled leaves cannot be restored the next morning), they are photographed and recorded, and the phenotype is observed and photographed after recovery culture at 28° C., as shown in FIG. 3A. The survival rate is calculated and the conductivity is measured. Survival rate=number of surviving plants/total number of plants x 100%; and the results are shown in FIG. 3B.
(iii) Conductivity measurement is as follows. The leaves of ZmRAFS transgenic maize plants and the control plant after heat shock recovery are taken for conductivity measurement. The leaves of the same leaf position are soaked in 15 mL of deionized water, vacuumed for 30 minutes, treated at 25° C. and 120 revolutions per minute (rpm) for 1 hour, and the conductivity is recorded as C₁. After the leaves are bathed in boiling water for 30 minutes and then cooled to room temperature, the conductivity (also referred to as ion leakage) is measured and recorded as C₂; C₁/C₂×100% is the relative conductivity of the leaves. The conductivity meter model is Thunder Magnet DDS-307; and the results are shown in FIG. 3C.

The above is only the illustrated embodiment of the disclosure, and the scope of protection of the disclosure is not limited to this. Any person skilled in the related art can obviously obtain simple changes or equivalent substitutions of the technical solution within the scope of the technology disclosed herein, which all belong to the scope of protection of the disclosure.

What is claimed is:

1. An application method of maize raffinose synthase (ZrnRAFS) gene in maize, comprising:
using the ZrnRAFS gene to improve heat stress tolerance of crops, wherein a sequence of a ZrnRAFS gene is shown in SEQ ID NO: 1.

2. The application method as claimed in claim 1, wherein a protein sequence encoded by the ZrnRAFS gene is shown in SEQ ID NO: 2.

3. The application method as claimed in claim 1, comprising:
regulating content of raffinose in leaves of the corps through manipulation of the ZmRAFS gene to thereby improve the heat stress tolerance of the crops.

4. The application method as claimed in claim 1, wherein the crop is maize.

5. The application method as claimed in claim 1, comprising:
extracting ribonucleic acid (RNA) from leaves of maize seedlings at a three-leaf stage and reverse transcribing into copy deoxyribonucleic acid (cDNA);
amplifying a coding region of the ZmRAFS gene by using the cDNA as a template with an upstream primer shown in SEQ ID NO: 3 and a downstream primer shown in SEQ ID NO: 4 to obtain an amplification product;
ligating the amplification product to a maize expression vector and transforming the ligated maize expression vector into an Agrobacterium strain; and
performing genetic transformation of maize on the transformed Agrobacterium strain to a maize transformation receptor, so as to obtain a target plant with the heat stress tolerance.

6. The application method as claimed in claim 5, wherein procedures of the amplifying comprise pre-denaturation at 95° C. for 5 minutes; 95° C. denaturation for 30 seconds, annealing at 60° C. for 30 seconds, extension at 72° C. for 1 minute and 20 seconds, 35 cycles; and final extension at 72° C. for 10 minutes.
